SEQUENCE LISTING (20 sequences)

SEQ ID NO: 1 - protein, 133 aa, Artificial Sequence
  Description of Artificial Sequence Recombinant protein
AEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 2 - protein, 241 aa, Artificial Sequence
  Description of Artificial Sequence Recombinant protein
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 3 - protein, 383 aa, Artificial Sequence
  Description of Artificial Sequence Recombinant protein
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIKGGGGSGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 4 - protein, 405 aa, Artificial Sequence
  Description of Artificial Sequence Recombinant protein
MKYLLPTAAAGLLLLAAQPAMAQVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIKGGGGSGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 5 - DNA, 1218 bp, Artificial Sequence
  Description of Artificial Sequence Recombinant DNA
atgaaatatctgctgccgaccgcagcagcgggtctgctgctgctggcagcacagcctgcaatggcacaggttaaactggaacagagcggtgccgaagttgttaaaccgggtgcaagcgttaaactgagctgtaaagcaagcggctttaacatcaaagatagctatatgcattggctgcgtcagggtccgggtcagcgtctggaatggattggttggattgatccggaaaatggtgataccgaatatgcaccgaaatttcagggtaaagcaacctttaccaccgataccagcgcaaataccgcatatctgggtctgagcagcctgcgtccggaagataccgcagtgtattattgtaatgaaggcaccccgaccggtccgtattatttcgattattggggtcagggcaccctggttaccgttagcagcggtggtggtggtagtggtggcggtggttcaggcggtggcggtagcgaaaatgttctgacccagagcccgagcagcatgagcgttagcgttggtgatcgtgttaatattgcatgtagcgcaagcagcagcgttccgtacatgcactggctgcagcagaaaccgggtaaaagcccgaaactgctgatttatctgaccagcaatctggcaagcggtgttccgagccgttttagcggtagcggtagtggcaccgattatagcctgaccattagcagcgtgcagcctgaagatgcagcaacctattattgtcagcagcgtagcagttatccgctgacctttggtggtggcaccaaactggaaattaaagggggtggtggctcaggtggcggaggtgcagaagcaggtattaccggtacatggtcagatcagctgggtgatacctttattgttaccgcaggcgcagatggtgcactgaccggcacctatgaaaatgcagttggtggtgcagaaagccgttatgtgctgaccggtcgttatgatagcgcaccggcaaccgatggtagcggcaccgcactgggttggaccgttgcatggaaaaataacagcaaaaatgcacatagcgcaaccacctggtcaggtcagtatgtgggtggtgccgatgccaaaattaacacccagtggctgctgaccagcggtacaaccaatgcaaatgcctggaaaagtaccctggttggtcatgatacattcaccaaagttaaaccgagcgcagcaagccatcatcatcaccatcattaa

SEQ ID NO: 6 - protein, 9 aa, Artificial Sequence
  Description of Artificial Sequence Recombinant linker
GGGGSGGGG

SEQ ID NO: 7 - DNA, 40 bp, Artificial Sequence
  Description of Artificial Sequence Synthesized DNA
aaggagatatacatatggataaaattgccattgttaatat

SEQ ID NO: 8 - DNA, 39 bp, Artificial Sequence
  Description of Artificial Sequence Synthesized DNA
ttgagatctgccatatgttatttcacttgtttcagaacg

SEQ ID NO: 9 - DNA, 35 bp, Artificial Sequence
  Description of Artificial Sequence Synthesized DNA
agaaggagatataccatgaaatatctgctgccgac

SEQ ID NO: 10 - DNA, 36 bp, Artificial Sequence
  Description of Artificial Sequence Synthesized DNA
cgccgagctcgaattttaatgatggtgatgatgatg

SEQ ID NO: 11 - DNA, 26 bp, Artificial Sequence
  Description of Artificial Sequence Synthesized DNA
ggtatatctccttcttaaagttaaac

SEQ ID NO: 12 - DNA, 24 bp, Artificial Sequence
  Description of Artificial Sequence Synthesized DNA
aattcgagctcggcgcgcctgcag

SEQ ID NO: 13 - DNA, 27 bp, Artificial Sequence
  Description of Artificial Sequence Synthesized DNA
agttaaataacgagctcggcgcgcctg

SEQ ID NO: 14 - DNA, 29 bp, Artificial Sequence
  Description of Artificial Sequence Synthesized DNA
gctcgttatttaactttggtgaatgtatc

SEQ ID NO: 15 - DNA, 1218 bp, Artificial Sequence; identical to SEQ ID NO: 5
  Description of Artificial Sequence Recombinant DNA

SEQ ID NO: 16 - protein, 405 aa, Artificial Sequence; identical to SEQ ID NO: 4
  Description of Artificial Sequence Recombinant protein

SEQ ID NO: 17 - DNA, 1185 bp, Artificial Sequence
  Description of Artificial Sequence Recombinant DNA
atgaaatatctgctgccgaccgcagcagcgggtctgctgctgctggcagcacagcctgcaatggcacaggttaaactggaacagagcggtgccgaagttgttaaaccgggtgcaagcgttaaactgagctgtaaagcaagcggctttaacatcaaagatagctatatgcattggctgcgtcagggtccgggtcagcgtctggaatggattggttggattgatccggaaaatggtgataccgaatatgcaccgaaatttcagggtaaagcaacctttaccaccgataccagcgcaaataccgcatatctgggtctgagcagcctgcgtccggaagataccgcagtgtattattgtaatgaaggcaccccgaccggtccgtattatttcgattattggggtcagggcaccctggttaccgttagcagcggtggtggtggtagtggtggcggtggttcaggcggtggcggtagcgaaaatgttctgacccagagcccgagcagcatgagcgttagcgttggtgatcgtgttaatattgcatgtagcgcaagcagcagcgttccgtacatgcactggctgcagcagaaaccgggtaaaagcccgaaactgctgatttatctgaccagcaatctggcaagcggtgttccgagccgttttagcggtagcggtagtggcaccgattatagcctgaccattagcagcgtgcagcctgaagatgcagcaacctattattgtcagcagcgtagcagttatccgctgacctttggtggtggcaccaaactggaaattaaagggggtggtggctcaggtggcggaggtgcagaagcaggtattaccggtacatggtcagatcagctgggtgatacctttattgttaccgcaggcgcagatggtgcactgaccggcacctatgaaaatgcagttggtggtgcagaaagccgttatgtgctgaccggtcgttatgatagcgcaccggcaaccgatggtagcggcaccgcactgggttggaccgttgcatggaaaaataacagcaaaaatgcacatagcgcaaccacctggtcaggtcagtatgtgggtggtgccgatgccaaaattaacacccagtggctgctgaccagcggtacaaccaatgcaaatgcctggaaaagtaccctggttggtcatgatacattcaccaaagttaaataa

SEQ ID NO: 18 - protein, 394 aa, Artificial Sequence
  Description of Artificial Sequence Recombinant protein
MKYLLPTAAAGLLLLAAQPAMAQVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIKGGGGSGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVK

SEQ ID NO: 19 - protein, 122 aa, Artificial Sequence
  Description of Artificial Sequence Recombinant protein
AEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVK

SEQ ID NO: 20 - protein, 6 aa, Artificial Sequence
  Description of Artificial Sequence Synthetic 6xHis tag
HHHHHH

(19) United States
(12) Patent Application Publication
(10) Pub. No.: US 2022/0348681 A1
(43) Pub. Date: Nov. 3, 2022

(54) FUSED PROTEIN OF AN ANTIBODY WHICH RECOGNIZES CANCER-CELLS AND A STREPTAVIDIN MUTANT

(71) Applicants: THE UNIVERSITY OF TOKYO, Tokyo (JP); SAVID THERAPEUTICS INC., Tokyo (JP)

(72) Inventors: Toshiya TANAKA, Tokyo (JP); Tatsuhiko KODAMA, Tokyo (JP); Takefumi YAMASHITA, Tokyo (JP); Motomu KANAI, Tokyo (JP); Kenzo YAMATSUGU, Tokyo (JP); Toshifumi TATSUMI, Tokyo (JP); Takeshi KAWAMURA, Tokyo (JP); Akira SUGIYAMA, Tokyo (JP); Masanobu TSUKAGOSHI, Tokyo (JP); Yuzo TODA, Tokyo (JP); Junji NISHIGAKI, Tokyo (JP)

(73) Assignees: THE UNIVERSITY OF TOKYO, Tokyo (JP); SAVID THERAPEUTICS INC., Tokyo (JP)

(21) Appl. No.: 17/237,716

(22) Filed: Apr. 22, 2021

(51) Int. Cl. C07K 16/30; C07K 16/32; C07K 14/36; A61K 49/00; A61K 39/395; A61K 38/16

(52) U.S. Cl. CPC C07K 16/3007; C07K 16/32; C07K 14/36; A61K 49/0032; A61K 39/3955; A61K 38/164; A61K 38/00

(57) ABSTRACT

[Object] To provide a fusion protein of an antibody that recognizes cancer cells and a mutant streptavidin, which is for use in the treatment or diagnosis of cancer.

[Means for Solution] A fusion protein comprising a protein-recognizing substance and the amino acid sequence as set forth in SEQ ID NO: 19 that is fused with the protein-recognizing substance.

### TECHNICAL FIELD

The present invention relates to a fusion protein of a protein-recognizing substance (for example, an antibody that recognizes cancer cells) and a mutant streptavidin, and the use thereof.

### BACKGROUND ART

Avidin and biotin, or streptavidin and biotin have an extremely high affinity between them (Kd=10⁻¹⁵ to 10⁻¹⁴M). This is one of extremely strong interactions between two biomolecules. At present, the interaction between avidin/streptavidin and biotin has been widely applied in the field of biochemistry, molecular biology, or medicine. A drug delivery method and a pretargeting method, in which high binding ability between avidin/streptavidin and biotin is combined with an antibody molecule, have been devised. In connection with these studies, a mutant streptavidin with a reduced affinity for natural biotin and a biotin-modified dimer having a high affinity for the mutant streptavidin with a low affinity for natural biotin are reported in Patent Document 1.

### PRIOR ART DOCUMENTS

### Patent Documents

[Patent Document 1] International Publication WO2015/125820

### SUMMARY OF INVENTION

### Object to be Solved by the Invention

It is an object of the present invention to provide a fusion protein of an antibody that recognizes cancer cells and a mutant streptavidin, which is for use in the treatment or diagnosis of cancer. Further, it is another object of the present invention to provide a means for treating cancer or a means for diagnosing cancer, in which the above-described fusion protein is used.

### Means for Solving the Object

As a result of intensive studies directed towards achieving the above-described objects, the present inventor has selected an anti-CEA antibody as an antibody that recognizes cancer cells, and then, has prepared a fusion protein of the anti-CEA antibody and a mutant streptavidin, in which the amino acid at the C-terminus thereof is deleted. Thereafter, the present inventor has found that the above-described fusion protein has an affinity with a biotin-modified dimer, thereby completing the present invention.

Specifically, according to the present invention, the following inventions are provided.

<1> A fusion protein comprising a protein-recognizing substance and the amino acid sequence as set forth in SEQ ID NO: 19 that is fused with the protein-recognizing substance.
<1> The fusion protein according to <1>, wherein the protein-recognizing substance is a substance that recognizes cancer cells.
<3> The fusion protein according to <2>, wherein the antibody that recognizes cancer cells is an anti-CEA antibody or an anti-HER2 antibody.
<4> The fusion protein according to any one of <1>to <3>, which has the amino acid sequence as set forth in SEQ ID NO: 18.
<5> A nucleic acid encoding the amino acid sequence as set forth in SEQ 11) NO: 18.
<6> A cancer therapeutic agent or a cancer diagnostic agent, comprising the fusion protein according to any one of <1>to <4>.
<7> A kit for treating or diagnosing cancer, comprising (1) the fusion protein according to any one of <1>to <4>, and (2) a conjugate of a compound represented by the following formula (1) or a salt thereof and a diagnostic substance or a therapeutic substance:

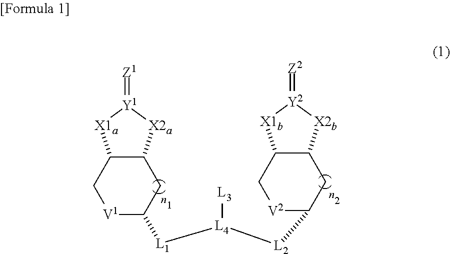

wherein
X1a, X1b, X2a and X2b each independently represent O or NH,
Y¹ and Y² each independently represent C or S,
Z¹ and Z² each independently represent O, S or NH,
V¹ and V² each independently represent S or S⁺—O⁻,
n1 and n2 each independently represent an integer of 0 or 1,
L₁ and L2 each independently represent a divalent linking group,
L₃ represents a group comprising a functional group capable of binding to the diagnostic substance or the therapeutic substance at the terminus, and
L₄ represents a trivalent linking group.
<8> The kit according to <7>, wherein the diagnostic substance or the therapeutic substance is a phthalocyanine dye.

### Advantageous Effects of Invention

The proliferation of cancer cells can be suppressed by using the fusion protein of the present invention consisting of an antibody that recognizes cancer cells and a mutant streptavidin.

### BRIEF DESCRIPTION OF DRAWINGS

FIG. 1 shows a CBB-stained SDS-PAGE electrophoretic pattern of the fusion protein of the present invention.

FIG. 2 shows evaluation of the binding performance of the fusion protein of the present invention with a modified biotin.

### EMBODIMENT OF CARRYING OUT THE INVENTION

Hereinafter, the present invention will be described in more detail.

### <Fusion Protein of Protein-Recognizing Substance and Mutant Streptavidin>

The fusion protein of the present invention comprises a protein-recognizing substance and the amino acid sequence as set forth in SEQ ID NO: 19 that is fused with the above-described protein-recognizing substance.

The protein-recognizing substance is preferably a substance that recognizes cancer cells.

As such a substance that recognizes cancer cells, an antibody, a peptide, a nucleic acid, an aptamer, etc., which target the following antigens specifically expressed in cancer, can be used:

Epiregulin, ROBO 1, 2, 3, and 4, 1-40-β-amyloid, 4-1BB, 5AC, 5T4, ACVR2B, adenocarcinoma antigen, α-fetoprotein, angiopoetin 2, anthrax toxin, AOC3 (VAP-1), B-lymphoma cells, B7-H3, BAFF, β amyloid, C242 antigen, C5, CA-125, carbonic anhydrase 9 (CA-DC), cardiac myosin, CCL11 (eotaxin-1), CCR4, CCR5, CD11, CD18, CD125, CD140a, CD147 (basigin), CD147 (basigin), CD15, CD152, CD154 (CD4OL), CD154, CD19, CD2, CD20, CD200, CD22, CD221, CD23 (IgE receptor), CD25 (IL-2 receptor α chain), CD28, CD3, CD30 (TNFRSF8), CD33, CD37, CD38 (cyclic ADP ribose hydrolase), CD4, CD40, CD41 (integrin α-IIb), CD44 v6, CD5, CD51, CD52, CD56, CD6, CD70, CD74, CD79B, CD80, CEA, CFD, ch4D5, CLDN18.2, Clostridium difficile, clumping factor A, CSF2, CTLA-4, cytomegalovirus, cytomegalovirus glycoprotein B, DLL4, DRS, E. coli Shiga toxin type 1, E. coli Shiga toxin type 2, EGFL7, EGFR, endotoxin, EpCAM, episialin, ERBB3, Escherichia coli, F protein of respiratory syncytial virus, FAP, fibrin II β chain, fibronectin extra domain-B, folate receptor 1, Frizzled receptor, GD2, GD3 ganglioside, GMCSF receptor a chain, GPNMB, hepatitis B surface antigen, hepatitis β virus, HER1, HER2/neu, HER3, HGF, HIV-1, HLA-DRβ, HNGF, Hsp90, human β amyloid, human scatter factor receptor kinase, human TNF, ICAM-1 (CD54), IFN-α, IFN-γ, IgE, IgE Fc region, IGF-1 receptor, IGF-I, IgG4, IGHE, IL-1β, IL-13, IL-17, IL-17A, IL-22, IL-23, IL-4, IL-5, IL-6, IL-6 receptor, IL-9, ILGF2, influenza A hemagglutinin, insulin-like growth factor I receptor, integrin α4, integrin α4β7, integrin α5β1, integrin α7β7, integrin αIIbβ3, integrin αVβ3, integrin γ induced protein, interferon receptor, interferon α/β receptor, ITGA2, ITGB2 (CD18), KIR2D, L-selectin (CD62L), Lewis-Y antigen, LFA-1 (CD11a), lipoteichoic acid, LOXL2, LTA, MCP-1, mesothelin, MS4A1, MUC1, mucin CanAg, myostatin, N-glycolylneuraminic acid, NARP-1, NCA-90 (granulocyte antigen), NGF, NOGO-A, NRP1, Oryctolagus cuniculus, OX-40, oxLDL, PCSK9, PD-1, PDCD1, PDGF-R α, phosphatidylserine, prostate cancer cells, Pseudomonas aeruginosa, Rabies virus glycoprotein, RANKL, respiratory syncytial virus, RHD, Rh (Rhesus) factor, RON, RTN4, sclerostin, SDC1, selectin P, SLAMF7, SOST, sphingosine- 1-phosphate, TAG-72, TEM1, tenascin C, TFPI, TGFβ1, TGFβ2, TGF-β, TNF-α, TRAIL-R1, TRAIL-R2, tumor antigen CTAA16.88, MUC1 tumor-specific glycosylation, TWEAK receptor, TYRP1 (glycoprotein 75), VEGF-A, VEGFR-1, VEGFR2, vimentin, and VWF.

The antibody that recognizes cancer cells is particularly preferably an anti-CEA antibody or an HER2 antibody.

An example of the fusion protein of the present invention is a fusion protein having the amino acid sequence as set forth in SEQ ID NO: 18.

According to the present invention, a nucleic acid (for example, DNA) encoding the fusion protein of the present invention is further provided.

The nucleic acid (for example, DNA) encoding the fusion protein of the present invention can be used by being incorporated into a vector. In order to produce the fusion protein of the present invention, a nucleic acid encoding the fusion protein of the present invention is incorporated into an expression vector, and a host is then transformed with this expression vector, so that the fusion protein of the present invention can be expressed in the host.

When Escherichia coli is used as a host, the vector preferably has a replication origin (ori) and also has a gene for selecting the transformed host (e.g. a drug-resistance gene that is resistant to drugs, such as ampicillin, tetracycline, kanamycin or chloramphenicol, etc.). Moreover, an expression vector preferably has a promoter capable of efficiently expressing the mutant streptavidin of the present invention in a host, such as a lacZ promoter or a T7 promoter. Examples of such a vector include an M13 vector, a pUC vector, pBR322, pBluescript, pCR-Script, pGEX-5X-1 (Pharmacia), “QlAexpress system” (QIAGEN), pEGFP, and pET (in this case, BL21 that expresses T7 RNA polymerase is preferably used as a host).

A vector can be introduced into a host cell by applying a calcium chloride method or an electroporation method, for example. Further, a sequence that encodes a tag for improving solubility, such as glutathione S-transferase, thioredoxin or a maltose-binding protein, may be added. Still further, a sequence that encodes a tag designed for facilitating purification, such as a polyhistidine tag, a Myc epitope, a hemagglutinin (HA) epitope, a T7 epitope, an Xpress tag, a FLAG tag or other known tag sequences, may also be added.

Other than Escherichia coli, examples of the expression vector include: mammal-derived expression vectors (for example, pcDNA3 (manufactured by Invitrogen), pEGF-BOS (Nucleic Acids. Res.1990, 18(17), p. 5322), pEF and pCDM8); insect cell-derived expression vectors (for example, “Bac-to-BAC baculovirus expression system” (manufactured by Gibco-BRL) and pBacPAK8); plant-derived expression vectors (for example, pMH1 and pMH2); animal virus-derived expression vectors (for example, pHSV, pMV and pAdexLcw); retrovirus-derived expression vectors (for example, pZlPneo); yeast-derived expression vectors (for example, “Pichia Expression Kit” (manufactured by Invitrogen), pNV11 and SP-Q01); and Bacillus subtilis-derived expression vectors (for example, pPL608 and pKTH50).

When the expression of the present fusion protein in an animal cell such as a CHO cell, a COS cell or an NIH3T3 cell is intended, it is essential for the expression vector to have a promoter necessary for the expression of the fusion protein in such an animal cell, such as an SV40 promoter (Mulligan et al., Nature (1979) 277, 108), an MMLV-LTR promoter, an EF 1 cc promoter (Mizushima et al., Nucleic Acids Res. (1990) 18, 5322) or a CMV promoter. It is more preferable if the expression vector has a gene for selecting the transformation of a cell (for example, a drug-resistance gene capable of determining transformation with the use of drugs (neomycin, G418, etc.)). Examples of a vector having such properties include pMAM, pDR2, pBK-RSV, pBK-CMV, pOPRSV and pOP13.

The type of a host cell, into which the vector is introduced, is not particularly limited. Either prokaryotes or eukaryotes may be used. It is possible to use Escherichia coli or various types of animal cells, for example.

In the case of using a eukaryotic cell, for example, an animal cell, a plant cell or a fungal cell can be used as a host. Examples of an animal cell that can be used herein include: mammalian cells such as a CHO cell, a COS cell, a 3T3 cell, a HeLa cell or a Vero cell; and insect cells such as Sf9, Sf21 or Tn5. When the expression of a large amount of the fusion protein in an animal cell is intended, a CHO cell is particularly preferable. A vector can be introduced into a host cell by a calcium phosphate method, a DEAE-dextran method, a method using cationic ribosome DOTAP (manufactured by Boehringer Mannheim), an electroporation method, a lipofection method or the like.

As a plant cell, a cell from Nicotiana tabacum has been known as a protein-producing system, for example. These cells may be subjected to callus culture. Examples of a known fungal cell include: yeast cells including genus Saccharomyces such as Saccharomyces cerevisiae; and filamentous fungi including genus Aspergillus such as Aspergillus niger.

Examples of a prokaryotic cell that can be used herein include Escherichia coli (E. coli), such as JM109, DH5α or BB101. Moreover, Bacillus subtilis has been known.

These cells are transformed with the nucleic acid of the present invention, and the transformed cells are then cultured in vitro, so as to obtain the fusion protein of the present invention. The culture can be carried out in accordance with a known culture method. Examples of a culture solution of animal cells that can be used herein include DMEM, MEM, RPMI1640, and IMDM. During the culture, a serum infusion such as fetal calf serum (FCS) may be used in combination, or serum free culture may also be carried out. The pH applied during the culture is preferably approximately pH 6 to 8. The culture is generally carried out at a temperature of approximately 30° C. to 40° C. for approximately 15 to 200 hours. As necessary, medium replacement, ventilation and stirring are carried out. Furthermore, growth factors may also be added to promote the growth of cells.

### <Cancer Therapeutic Agent or Cancer Diagnostic Agent>

The fusion protein of the present invention is useful as a cancer therapeutic agent or a cancer diagnostic agent.

According to the present invention, provided is a kit for treating or diagnosing cancer, comprising (1) the fusion protein of the present invention, and (2) a conjugate of a compound represented by a formula (1) as shown below or a salt thereof, and a diagnostic substance or a therapeutic substance.

By administering the fusion protein of the present invention to a patient, a mutant streptavidin can be accumulated in the body of the patient, specifically into cancer cells. Subsequently, by administering a conjugate of a biotin-modified dimer having an affinity for the mutant streptavidin and a diagnostic substance or a therapeutic substance to the patient, it becomes possible to accumulate the diagnostic substance or the therapeutic substance precisely into the cancer cells.

Otherwise, a complex is prepared by binding the “fusion protein of the present invention” with the “conjugate of a biotin-modified dimer having an affinity for a mutant streptavidin and a diagnostic substance or a therapeutic substance,” and the thus prepared complex can be administered to the patient.

### <Biotin-Modified Dimer>

The biotin-modified dimer is a compound represented by the following formula (1) or a salt thereof, and is preferably a compound represented by the following formula (2) or a salt thereof. As such a biotin-modified dimer, the compound described in International Publication WO2015/125820 can be used.

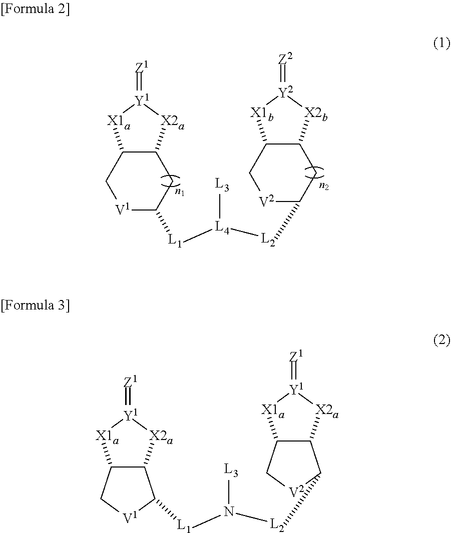

wherein, in the above formulae,
X1a, X1b, X2a and X2b each independently represent O or NH,
Y¹ and Y² each independently represent C or S,
Z¹ and Z² each independently represent O, S or NH,
V¹ and V² each independently represent S or S⁺O⁻,
n1 and n2 each independently represent an integer of 0 or 1,
L₁ and L₂ each independently represent a divalent linking group,
L₃ represents a group comprising a functional group capable of binding to the diagnostic substance or the therapeutic substance (for example, a phthalocyanine dye) at the terminus, and
L₄ represents a trivalent linking group.

In the formula (1) and the formula (2), the moieties represented by the following structures:

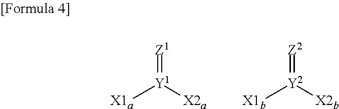

are preferably any one of the following moieties, but are not limited thereto:

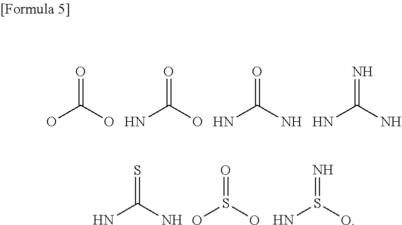

X1a, X1b, X2a and X2b preferably represent NH; Y¹ and Y² preferably represent C; Z¹ and Z² preferably represent NH; and V¹ and V² preferably represent S.

L₁ and L₂ each independently represent a divalent linking group consisting of a combination of groups selected from —CONH—, —NHCO—, —COO—, —OCO—, —CO—, —O—, and an alkylene group containing 1 to 10 carbon atoms.

Preferably, L₁ and L₂ each independently represent a divalent linking group consisting of a combination of groups selected from —CONH—, —NHCO—, —O—, and an alkylene group containing 1 to 10 carbon atoms.

Preferably, L₁ and L₂ each independently represent a divalent linking group consisting of a combination of groups selected from —CONH—, —NHCO—, and an alkylene group containing 1 to 10 carbon atoms.

L₄ represents a trivalent linking group, and is preferably the following:

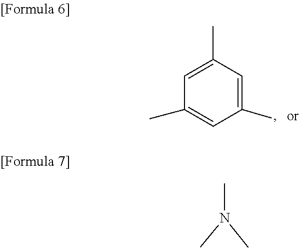

(which is a benzene-derived trivalent linking group or a nitrogen atom).

L₃ is preferably a group consisting of a combination of groups selected from —CONH—, —NHCO—, —COO—, —OCO—, —CO—, —O—, and an alkylene group containing 1 to 10 carbon atoms, and further comprising an amino group at the terminus.

<Conjugate of Biotin-Modified Dimer and Diagnostic Substance or Therapeutic Substance>

By binding a diagnostic substance or a therapeutic substance to a biotin-modified dimer, a conjugate of the biotin-modified dimer and the diagnostic substance or the therapeutic substance can be prepared. Examples of the diagnostic substance or the therapeutic substance may include a fluorochrome, a chemiluminescent agent, a radioisotope, a sensitizer consisting of a metal compound or the like, a neutron-capturing agent consisting of a metal compound or the like, a phthalocyanine dye, a low-molecular-weight compound, micro- or nano-bubbles, and a protein. Preferably, a phthalocyanine dye can be used.

### <Phthalocyanine Dye>

The phthalocyanine dye is preferably a silicon phthalocyanine dye. Specific examples of the phthalocyanine dye, such as 1RDye (registered trademark) 700DX, are described in, for example, U.S. Pat. No. 7,005,518.

As such a phthalocyanine dye, a dye represented by the following formula (21) can be used. As an example, a dye represented by the following formula (22) can be used.

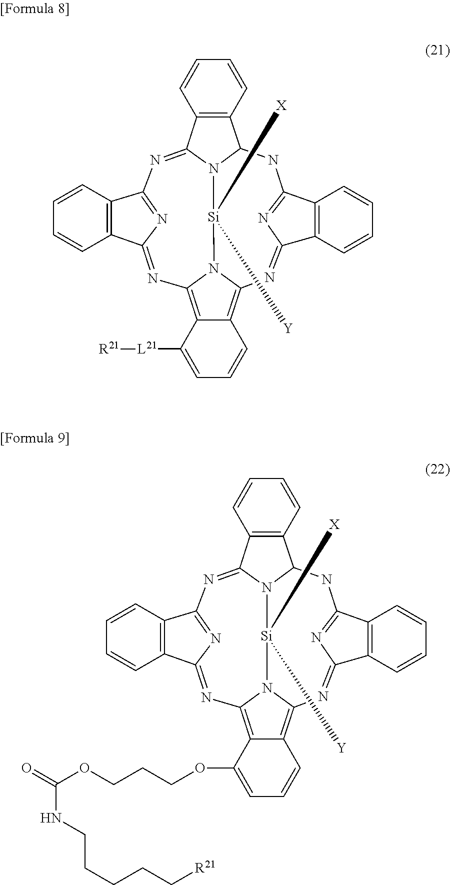

In the above formulae, L²¹ represents a divalent linking group, R²¹ represents a functional group capable of binding to the compound represented by the formula (1) or a salt thereof.

In the above formulae, X and Y each independently represent a hydrophilic group, —OH, a hydrogen atom, or a substituent. Examples of the substituent used herein may include, but are not particularly limited to, a halogen atom (a fluorine atom), a substituent containing a carbon atom (a hydrocarbo group, etc.), and a substituent containing a nitrogen atom (an amino group, etc.).

Specific examples of X and Y may include:

(i) a case where both X and Y are hydrophilic groups;
(ii) a case where either X or Y is a hydrophilic group, and the other is —OH or a hydrogen atom; and
(iii) a case where X and Y are —OH or hydrogen atoms.

The hydrophilic group(s) represented by X and/or Y are not particularly limited. One example is shown below.

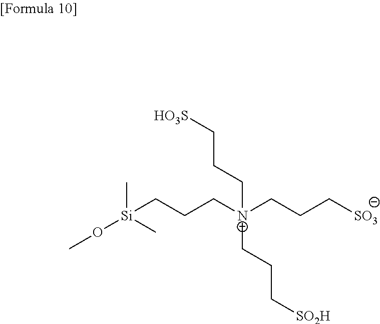

As a phthalocyanine dye, a commercially available product such as IRDye (registered trademark) 700DX can be used. In the present invention, an NHS ester of IRDye (registered trademark) 700DX is used, and is allowed to react with a biotin-modified dimer having an amino group to produce a conjugate. Other variations of IRDye (registered trademark) 700DX are described in U.S. Pat. No. 7,005,518, and those can also be used.

R²¹ represents a functional group capable of binding to the compound represented by the formula (1) or a salt thereof. R²¹ is preferably a functional group that can react with a carboxyl group, amine or a thiol group on the biotin-modified dimer and can bind thereto. Preferred examples of R²¹ may include, but are not particularly limited to, activated ester, halogenated acyl, halogenated alkyl, appropriately substituted amine, anhydride, carboxylic acid, carbodiimide, hydroxyl, iodoacetamide, isocyanate, isothiocyanate, maleimide, NHS ester, phosphoramidite, sulfonic acid ester, thiol, and thiocyanate.

L²¹ represents a divalent linking group, and for example, it may include: any given combination of an ether, thioether, amine, ester, carbamate, urea, thiourea, oxy or amide bond, or a single, double, triple or aromatic carbon-carbon bond; or a phosphorus-oxygen, phosphorus-sulfur, nitrogen-nitrogen, nitrogen-oxygen, or nitrogen-platinum bond; or an aromatic or heteroaromatic bond. L²¹ is preferably a group consisting of a combination of groups selected from —CONH—, —NHCO—, —COO—, —OCO—, —CO—, —O—, and an alkylene group containing 1 to 10 carbon atoms.

-L²¹-R² may include a phosphoramidite group, NHS ester, active carboxylic acid, thiocyanate, isothiocyanate, maleimide, and iodoacetamide.

L²¹ represents a —(CH₂)ₙ-group, and in the formula, n is an integer of 1 to 10, and is preferably an integer of 1 to 4. As one example, L²¹-R²¹ is —O—(CH₂)₃—OC(O)NH—(CH₂)₅—C(O)O—N-succinimidyl.

### <Photoimmunotherap >

Photoimmunotherapy is a therapeutic method of using a photosensitizer and an irradiation light to destroy specific cells in a body. When a photosensitizer is exposed to a light with a specific wavelength, it generates cytotoxic reactive oxygen species capable of inducing apoptosis, necrosis, and/or autophagy to around cells. For example, Japanese Patent No. 6127045 discloses a method of killing cells, comprising: a step of allowing cells comprising a cell surface protein to come into contact with a therapeutically effective amount of one or more antibodies-IR700 molecules, wherein the antibodies specifically bind to the cell surface protein; a step of irradiating the cells with a light at a wavelength of 660 to 740 nm and at a dose of at least 1 Jcm⁻²; and a step of allowing the cells to come into contact with one or more therapeutic agents at approximately 0 to 8 hours after the irradiation, thereby killing the cells. JP Patent Publication (Kohyo) No. 2017-524659 A discloses a method of inducing cytotoxicity to a subject affected with a disease or a pathology, comprising: (a) administering to a subject, a therapeutically effective drug comprising a phthalocyanine dye such as IRDye (registered trademark) 700DX conjugated with a probe specifically binding to the cell of the subject; and (b) irradiating the cell with an appropriate excitation light in an amount effective for inducing cell death.

The fusion protein of the present invention and the conjugate of a biotin-modified dimer and a phthalocyanine dye are administered to a subject, and the cells are then irradiated with an excitation light in an amount effective for suppression of cell proliferation or induction of cell death, so that the cell proliferation can be suppressed or the cell death can be induced, and thereby the subject can be treated.

Preferably, the fusion protein of the present invention and the conjugate of a biotin-modified dimer and a phthalocyanine dye are administered to a subject, and the cells are then irradiated with an excitation light in an amount effective for suppression of cell proliferation or induction of cell death, so that the cell proliferation can be suppressed or the cell death can be induced, and thereby the subject can be treated.

The subject used herein includes humans and non-human animals. Examples of the subject may include humans and experimental animals such as mice. The subject is preferably affected with a disease regarding which suppression of cell proliferation or induction of cell death is desired. For example, the subject is affected with cancer or solid tumor.

Examples of the “cancer” may include carcinoma, lymphoma, blastoma, sarcoma, and leukemia or malignant lymphoma. Specific examples of the cancer may include squamous cell carcinoma (e.g., epithelial squamous cell carcinoma), lung cancer including small cell lung cancer, non-small cell lung cancer (“NSCLC”), pulmonary adenocarcinoma and pulmonary squamous cell carcinoma, peritoneal cancer, hepatocarcinoma, corpus ventriculi or stomach cancer, including digestive cancer, pancreatic cancer, glioblastoma, cervical cancer, ovarian cancer, liver cancer, bladder cancer, hepatocellular cancer, breast cancer, colon cancer, rectal cancer, colorectal cancer, endometrial membrane cancer or endometrial carcinoma, salivary gland carcinoma, kidney or renal region cancer, prostate cancer, vulvar cancer, thyroid cancer, hepatocellular carcinoma, anal carcinoma, penile carcinoma, and head and neck cancer.

The solid tumor means a benign or malignant, abnormal cell mass that generally does not contain a capsule. Examples of the solid tumor may include glioma, astrocytoma, medulloblastoma, craniopharyngioma, ependymoma, pineal gland tumor, hemangioblastoma, acoustic neuroma, oligodendrocyte, meningioma, melanoma, neuroblastoma, and retinoblastoma.

Examples of the administration method to the subject may include, but are not limited to, a local route, an injection (a subcutaneous injection, an intramuscular injection, an intradermal injection, an intraperitoneal injection, an intratumoral injection, an intravenous injection, etc.), an oral route, an ocular route, a sublingual route, a rectal route, a percutaneous route, an intranasal route, a vaginal route, and an inhalation route.

It is preferable that the conjugate of a biotin-modified dimer and a phthalocyanine dye and the fusion protein of the present invention are each administered in a therapeutically effective amount.

Regarding each of the above-described conjugate and fusion protein, the therapeutically effective amount per 60 kg is at least 0.5 mg (mg/60 kg), at least 5 mg/60 kg, at least 10 mg/60 kg, at least 20 mg/60 kg, at least 30 mg/60 kg, or at least 50 mg/60 kg. For example, when it is intravenously administered, the applied dose is 1 mg/60 kg, 2 mg/60 kg, 5 mg/60 kg, 20 mg/60 kg, or 50 mg/60 kg, and it is, for example, 0.5 to 50 mg/60 kg. In another example, the therapeutically effective amount is at least 100 μg/kg, at least 500 μg/kg or at least 500 μg/kg, and it is, for example, at least 10 μg/kg. For example, when it is intratumorally or intraperitoneally administered, the dose is 100 μg/kg, 250 μg/kg, approximately 500 μg/kg, 750 μg/kg, or 1000 μg/kg, and it is, for example, 10 μg/kg to 1000 μg/kg. In one example, when it is administered in the form of a solution for local administration, the therapeutically effective amount is 10 μg/ml, 20 μg/ml, 30 μg/ml, 40 μg/ml, 50 μg/ml, 60 μg/ml, 70 μg/ml, 80 μg/ml, 90 μg/ml, 100 μg/ml or the like, or it is 20 μg/ml to 100 μg/ml, or it is at least 500 μg/ml, or at least 1 μg/ml.

The above-described dose can be administered once or divided doses over several administrations (2, 3, or 4 times, etc.), or as a single preparation.

The conjugate of a biotin-modified dimer and a phthalocyanine dye and the fusion protein of the present invention can be each administered alone, or can also be administered in the presence of a pharmaceutically acceptable carrier, or can also be administered in the presence of other therapeutic agents (other anticancer agents, etc.).

The conjugate of a biotin-modified dimer and a phthalocyanine dye and the fusion protein of the present invention can bind to target cells or target tissues, such as circulating tumor cells or solid tumor cells. Thereafter, the target cells or tissues are irradiated with a light, so that the above-described conjugate or complex can absorb the light and can damage or destroy the target cells or tissues.

In the photoimmunotherapy, the wavelength of the irradiation light is preferably 660 to 740 nm, and the irradiation light has a wavelength of, for example, 660 nm, 670 nm, 680 nm, 690 nm, 700 nm, 710 nm, 720 nm, 730 nm, or 740 nm. Light irradiation may be carried out using a device equipped with a near infrared (NIR) light emitting diode.

The light irradiation amount is at least 1 J/cm², for example, at least 4 J/cm², at least 10 J/cm², at least 15 J/cm², at least 20 J/cm², at least 50 J/cm², or at least 100 J/cm². It is, for example, 1 to 500 J/cm². Light irradiation may be carried out several times (e.g., 2, 3, 4, 5, 6, 7, 8, 9, or 10 times).

The entire contents disclosed in Japanese Patent Application No. 2019-179280 filed on Sep. 30, 2019, are incorporated herein by reference in their entireties.

The present invention will be more specifically described in the following examples. However, these examples are not intended to limit the scope of the present invention.

### EXAMPLES

### Synthesis Example 1: 2-((4-(5-((3aS,4S,6aR)-2-Iminiohexahydro-1H-thieno[3,4-d]imidazol-4-yl)pentanamido)butypamino)-N-(2-((4-(5-((3aS,4S,6aR)-2-iminiohexahydro-1H-thieno[3,4-d]imidazol-4-yl)pentanamido)butypamino)-2-oxoethyl)-N-(2-(2-(2-(3-iodobenzamido)ethoxy)ethoxy)ethyl)-2-oxoethan-1-aminium) 2,2,2-trifluoroacetate

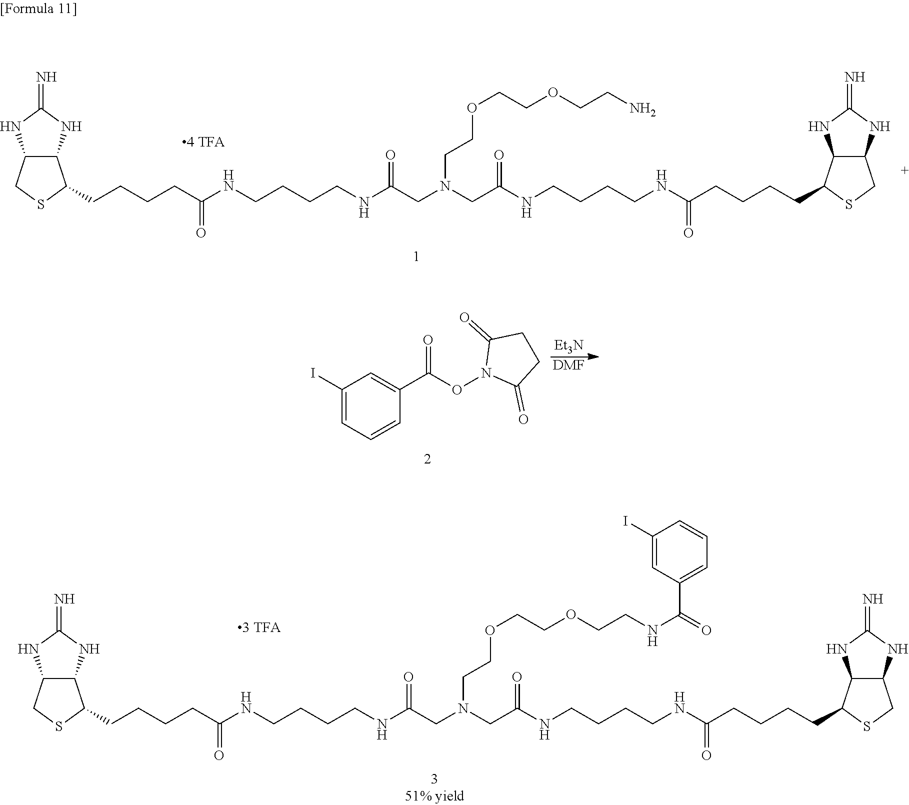

Compound 2 (0.7 mg, 2.06 μmol) dissolved in DMF (100 _{I}d) and triethylamine (4.3 μ1, 30.9 μmol) were added to the bisiminobiotin 1 (2.7 mg, 2.06 μmol in a DMF (100 μl) solution at room temperature. The obtained mixture was stirred in an argon atmosphere at room temperature for 5 hours, and the solvent was then removed under reduced pressure. The obtained crude product was purified by reversed phase HPLC (gradient: 0% for 5 min; 0-100% for 90 min CH₃CN in a 0.1% CF₃COOH aqueous solution, retention time: 41.5 min, YMC-Triart C18, flow rate=3.5 ml/min), so as to obtain a target compound 3 (1.5 mg, a yield of 51%). LRMS (ESI): m/z 362 [M+3H]³⁺.

The target compound 3 is also referred to as “Psyche J-iodine.”

### Example 1: Expression and Purification of Protein

V2122 is the mutant streptavidin described in Example 3 of International Publication WO2015/125820 (SEQ ID NO: 4 shown in International Publication WO2015/125820). The amino acid sequence of V2122 (a sequence having a 6xHis tag at the C-terminus) is as set forth in SEQ ID NO: 1 in the sequence listing.

scFv-V2122 is prepared by binding a single-chain antibody (scFv) against CEACAMS with the above-described V2122. This scFv-type anti-CEACAMS antibody is an scFv sequence described in a patent document U.S. Pat. No. 7,626,011B2. The amino acid sequence of the scFv-type anti-CEACAMS antibody is as set forth in SEQ ID NO: 2 in the sequence listing. In addition, the amino acid sequence of CEA-V2122 prepared by binding the scFv-type anti-CEACAM5 antibody with V2122 via an amino acid linker (GGGGSGGGG) (SEQ 11) NO: 6) is as set forth in SEQ ID NO: 3 in the sequence listing.

For the expression of a CEA-V2122 fusion protein, the DNA codon of a CEA-V2122 gene sequence, in which a pelB signal for secretion and expression in Escherichia coli had been incorporated into the N-terminus and a 6xHis-Tag sequence had been incorporated into the C-terminus, was optimized for Escherichia coli, thereby synthesizing an artificial gene. This amino acid sequence is as set forth in SEQ ID NO: 4 in the sequence listing, and the DNA sequence is as set forth in SEQ ID NO: 5 in the sequence listing.

As a specific protein expression vector, a vector prepared by incorporating a chaperone skp gene into MCS2 of a pETDuet1 vector was used. Regarding the skp gene, the DNA codon was optimized for Escherichia coli based on the amino acid sequence as set forth in SEQ ID NO: 6 in the sequence listing, thereby synthesizing an artificial gene. The synthesized skp gene was amplified by PCR, using the primers (AAGGAGATATACATATGGATAAAATTGCCATTGTTAATAT (SEQ 11) NO: 7), and TTGAGATCTGCCATATGTTATTTCACTTGTTTCAGAACG (SEQ ID NO: 8)), and the amplified gene was then cloned into MCS2 of the pETDue1 vector linearized with the restriction enzyme NdeI, using In-Fusion HD Cloning Kit, so as to obtain pETDuet_skp. Subsequently, the CEA-V2122 gene was incorporated into MCS1 of pETDuet_skp. Specifically, the artificially synthesized CEA-V2122 gene was amplified by PCR, using the primers (AGAAGGAGATATACCATGAAATATCTGCTGCCGAC (SEQ ID NO: 9), and CGCCGAGCTCGAATTTTAATGATGGTGATGATGATG (SEQ ID NO: 10)). Moreover, pETDuet_skp was linearized by PCR, using the primers (GGTATATCTCCTTCTTAAAGTTAAAC (SEQ ID NO: 11), and AATTCGAGCTCGGCGCGCCTGCAG (SEQ ID NO: 12)). The CEA-V2122 amplified by PCR and the linearized pETDuet_skp were subjected to cloning using In-Fusion HD Cloning Kit. The cloned vector was confirmed by sequencing, in terms of the gene sequence incorporated therein, and thereafter, it was referred to as pETDuet_CEA-V2122_skp.

CEA-V2122 de15 has an amino acid sequence obtained by using pETDuet_CEA-V2122_skp as a template, changing the codon encoding the 11th amino acid from the C-terminus to a stop codon according to PCR, and then removing 11 amino acids including 6xHis tag. Specifically, using the primers (AGTTAAATAACGAGCTCGGCGCGCCTG (SEQ ID NO: 13) and GCTCGTTAITTAACITTGGTGAATGTATC (SEQ ID NO: 14)), a mutation was introduced into pETDuet_CEA-V2122_skp used as a template according to PCR, so that the codon encoding the 11th amino acid from the C-terminus was changed to a stop codon. The obtained PCR product was treated with the restriction enzyme Dnp I according to a common method, and thereafter, competent cells DH5a (TOYOBO) were transformed with the PCR product. The transformed Escherichia coli was seeded on an LB plate, and on the following day, a colony was inoculated into 2 mL of an LB medium, followed by performing culture. Using Miniprep Kit (QIAGEN), a plasmid was prepared and was then sequenced, so that the introduction of the mutation was confirmed. The gene sequence before the introduction of the mutation, comprising a pelB signal sequence for the expression of a periplasmic space, is as set forth in SEQ ID NO: 15, and the amino acid sequence thereof is as set forth in SEQ ID NO: 16; and the gene sequence after the introduction of the mutation is as set forth in SEQ ID NO: 17, and the amino acid sequence thereof is as set forth in SEQ ID NO: 18. In addition, the plasmid vector, into which the mutation is introduced, is defined as pETDuet_CEA-V2122-del5_skp. An amino acid sequence that corresponds to the amino acid sequence of pETDuet_CEA-V2122_ skp as set forth in SEQ ID NO: 18, in which the 11 amino acids including the 6xHis tag at the C-terminus are removed from V2122, is as set forth in SEQ ID NO: 19.

For the expression of the protein, pETDuet_CEA-V2122-del5_skp was transformed into BL21(DE3) (Nippon Gene Co., Ltd.), which was then pre-cultured in 2xYT medium (SIGMA-ADLRICH) at 37° C. overnight. The medium used in the pre-culture was added to a new medium for 100-fold dilution, and culture was then carried out at 37° C., until OD (600 nm) became 0.5 to 2.0. Subsequently, IPTG was added to the culture to a final concentration of 0.5 mM, and the obtained mixture was then cultured at 37° C. for 4 hours. Thereafter, a culture supernatant was recovered and was then preserved at 4° C.

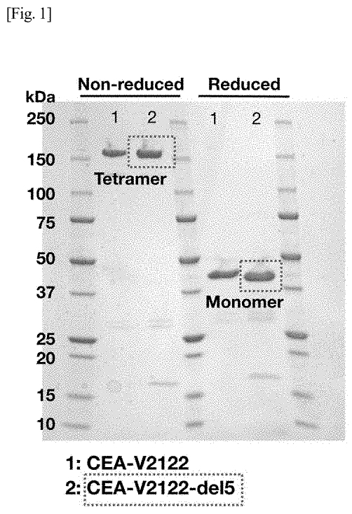

The recovered culture supernatant was purified using a Protein L column. Specifically, 1 mL of Capto L (GE Healthcare Life Sciences) was filled into a PD-10 column, and was then equilibrated with 10 column volume of PBS, and the aforementioned roughly purified product was then applied thereto. Thereafter, the resultant was washed with 10 column volume of PBS, was then eluted with 10 mM glycine hydrochloride (pH 2.0), and was then subjected to centrifugal concentration using Vivaspin Turbo 15 (MWCO 100,000). Moreover, using PD-10 (GE Healthcare Life Science), the buffer was replaced with PBS, and centrifugal concentration was further carried out using Vivaspin Turbo 4 (MWCO 100,000) to obtain a finally purified product. CEA-V2122 and CEA-V2122-del5, which had also been expressed and purified, were subjected to SDS-PAGE electrophoresis, and were then subjected to CBB staining. The results are shown in FIG. 1. As an SDS-PAGE gel, Mini-PROTEAN TGX 4-15% (Bio-Rad) was used, and as a CBB staining solution, Bullet CBB Stain One (Ready To Use) (Nacalai Tesque, Inc.) was used.

### Example 2: Analysis of Interaction Between CEA-V2122-del5 and Modified Biotin

The interaction between CEA-V2122-del5 and a modified biotin was analyzed using Biacore T200. The Psyche J-iodine described in Production Example 1 was used herein as a modified biotin.

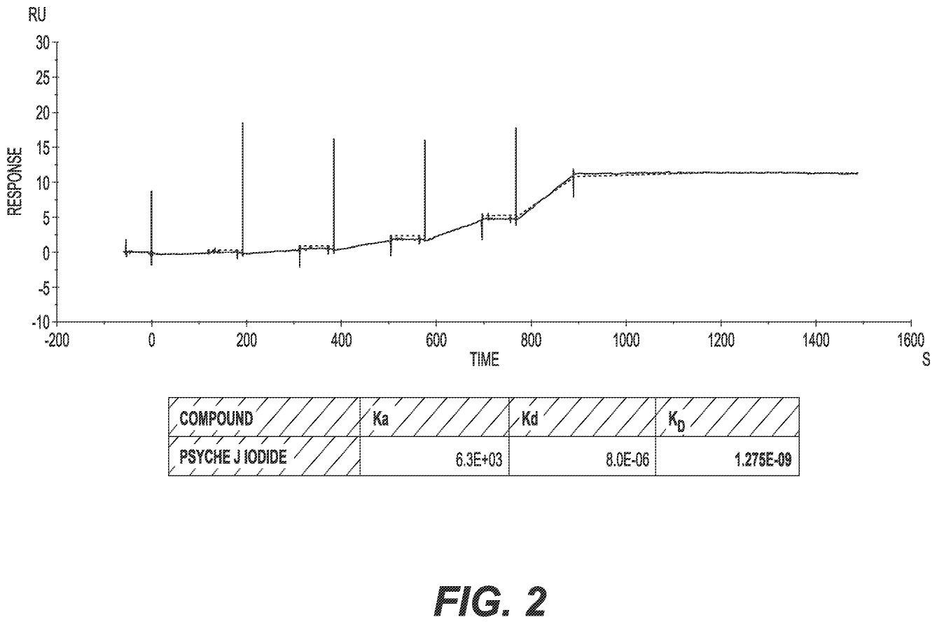

The analysis method was specifically as follows. That is, an amine-coupling kit was used, a target value was set to be 3000 RU, and the purified CEA-V2122-del5 was immobilized on Sensor Chip CM5. With regard to the concentrations of the analytes, 2.5E−09 M was two-fold diluted, and 5 types of two-fold serial dilutions including 2.5E-09 M were prepared and used in the measurement. Regarding interaction analysis, data were obtained by single-cycle kinetics analysis. Using Biacore T200 Evaluation Software, version 2.0, the obtained data were subjected to curve fitting in a bivalent analysis mode, and the following values were obtained: ka1=6.3E+03, and kd1=8.0E−06. Moreover, since evaluation can be carried out at K_{D}=kd1/ka1 in the bivalent analysis, the evaluation value K_{D}=kd1/ka1=6.3E+03/8.0E−06=1.3E−09 was obtained. These results are shown in FIG. 2.

From the Ki_{D} value in the sensorgram shown in FIG. 2, it was confirmed that CEA-V2122-del5 strongly binds to a modified biotin.

SEQ ID NO: 1
AEAGITGTWSDQLGDTFIVTAGADGALTGTYENAVGGAESRYVLTGRYDS
APATDGSGTALGWTVAWKNNSKNAHSATTWSGQYVGGADAKINTQWLLTS
GTTNANAWKSTLVGHDTFTKVKPSAASHHHHHH
(sm3E-scFv sequence)
SEQ ID NO: 2
QVKLEQSGAEVVKPGASVKLSCKASGFNIKDSYMHWLRQGPGQRLEWIGW
IDPENGDTEYAPKFQGKATFTTDTSANTAYLGLSSLRPEDTAVYYCNEGI
PTGPYYFDWGQGTLVTVSSGGGGSGGGGSGGGGSENVLTQSPSSMSVSVG
DRVNIACSASSSVPYMHWLQQKPGKSPKLLIYLTSNLASGVPSRFSGSGS
GTDYSLTISSVQPEDAATYYCQQRSSYPLTFGGGTKLEIK
SEQ ID NO: 3
QVKLEQSGAEVVKPGASVKLSCKASGFNIKDSYMHWLRQGPGQRLEWIGW
IDPENGDTEYAPKFQGKATFTTDTSANTAYLGLSSLRPEDTAVYYCNEGI
PTGPYYFDWGQGTLVTVSSGGGGSGGGGSGGGGSENVLTQSPSSMSVSVG
DRVNIACSASSSVPYMHWLQQKPGKSPKLLIYLTSNLASGVPSRFSGSGS
GTDYSLTISSVQPEDAATYYCQQRSSYPLTFGGGTKLEIKGGGGSGGGGA
EAGITGTWSDQLGDTFIVTAGADGALTGTYENAVGGAESRYVLTGRYDSA
PATDGSGTALGWTVAWKNNSKNAHSATTWSGQYVGGADAKINTQWLLTSG
TTNANAWKSTLVGHDTFTKVKPSAASHHHHHH
SEQ ID NO: 4
MKYLLPTAAAGLLLLAAQPAMAQVKLEQSGAEVVKPGASVKLSCKASGFN
IKDSYMHWLRQGPGQRLEWIGWIDPENGDTEYAPKFQGKATFTTDTSANT
AYLGLSSLRPEDTAVYYCNEGTPTGPYYFDWGQGTLVTVSSGGGGSGGGG
SGGGGSENVLTQSPSSMSVSVGDRVNIACSASSSVPYMHWLQQKPGKSPK
LLIYLTSNLASGVPSRFSGSGSGTDYSLTISSVQPEDAATYYCQQRSSYP
LTFGGGTKLE1KGGGGSGGGGAEAGITGTWSDQLGDTFIVTAGADGALTG
TYENAVGGAESRYVLTGRYDSAPATDGSGTALGWTVAWKNNSKNAHSATT
WSGQYVGGADAKINTQWLLTSGTTNANAWKSTLVGHDTFTKVKPSAASHH
HHHH
SEQ ID NO: 5
ATGAAATATCTGCTGCCGACCGCAGCAGCGGGTCTGCTGCTGCTGGCAGC
ACAGCCTGCAATGGCACAGGTTAAACTGGAACAGAGCGGTGCCGAAGTTG
TTAAACCGGGTGCAAGCGTTAAACTGAGCTGTAAAGCAAGCGGCTTTAAC
ATCAAAGATAGCTATATGCATTGGCTGCGTCAGGGTCCGGGTCAGCGTCT
GGAATGGATTGGTTGGATTGATCCGGAAAATGGTGATACCGAATATGCAC
CGAAATTTVAGGGTAAAGCAACCTTTACCACCGATACCAGCGCAAATACC
GCATATCTGGGTCTGAGCAGCCTGCGTCCGGAAGATACCGCAGTGTATTA
TTGTAATGAAGGCACCCCGACCGGTCCGTATTATTTCGATTATTGGGGTC
AGGGCACCCTGGTTACCGTTAGCAGCGGTGGTGGTGGTAGTGGTGGCGGT
GGTTCAGGCGGTGGCGGTAGCGAAAATGTTCTGACCCAGAGCCCGAGCAG
CATGAGCGTTAGCGTTGGTGATCGTGTTAATATTGCATGTAGCGCAAGCA
GCAGCGTTCCGTACATGCACTGGCTGCAGCAGAAACCGGGTAAAAGCCCG
AAACTGCTGATTTATCTGACCAGCAATCTGGCAAGCGGTGTTCCGAGCCG
TTTTAGCGGTAGCGGTAGTGGCACCGATTATAGCCTGACCATTAGCAGCG
TGCAGCCTGAAGATGCAGCAACCTATTATTGTCAGCAGCGTAGCAGTTAT
CCGCTGACCTTTGGTGGTGGCACCAAACTGGAAATTAAAGGGGGTGGTGG
CTCAGGTGGCGGAGGTGCAGAAGCAGGTATTACCGGTACATGGTCAGATC
AGCTGGGTGATACCTTTATTGTTACCGCAGGCGCAGATGGTGCACTGACC
GGCACCTATGAAAATGCAGTTGGTGGTGCAGAAAGCCGTTATGTGCTGAC
CGGTCGTTATGATAGCGCACCGGCAACCGATGGTAGCGGCACCGCACTGG
GTTGGACCGTTGCATGGAAAAATAACAGCAAAAATGCACATAGCGCAACC
ACCTGGTCAGGTCAGTATGTGGGTGGTGCCGATGCCAAAATTAACACCCA
GTGGCTGCTGACCAGCGGTACAACCAATGCAAATGCCTGGAAAAGTACCC
TGGTTGGTCATGATACATTCACCAAAGTTAAACCGAGCGCAGCAAGCCAT
CATCATCACCATCATTAA
SEQ ID NO: 15
ATGAAATATCTGCTGCCGACCGCAGCAGCGGGTCTGCTGCTGCTGGCAGC
ACAGCCTGCAATGGCACAGGTTAAACTGGAACAGAGCGGTGCCGAAGTTG
TTAAACCGGGTGCAAGCGTTAAACTGAGCTGTAAAGCAAGCGGCTTTAAC
ATCAAAGATAGCTATATGCATTGGCTGCGTCAGGGTCCGGGTCAGCGTCT
GGAATGGATTGGTTGGATTGATCCGGAAAATGGTGATACCGAATATGCAC
CGAAATTTCAGGGTAAAGCAACCTTTACCACCGATACCAGCGCAAATACC
GCATATCTGGGTCTGAGCAGCCTGCGTCCGGAAGATACCGCAGTGTATTA
TTGTAATGAAGGCACCCCGACCGGTCCGTATTATTTCGATTATTGGGGTC
AGGGCACCCTGGTTACCGTTAGCAGCGGTGGTGGTGGTAGTGGTGGCGGT
GGTTCAGGCGGTGGCGGTAGCGAAAATGTTCTGACCCAGAGCCCGAGCAG
CATGAGCGTTAGCGTTGGTGATCGTGTTAATATTGCATGTAGCGCAAGCA
GCAGCGTTCCGTACATGCACTGGCTGCAGCAGAAACCGGGTAAAAGCCCG
AAACTGCTGATTTATCTGACCAGCAATCTGGCAAGCGGTGTTCCGAGCCG
TTTTAGCGGTAGCGGTAGTGGCACCGATTATAGCCTGACCATTAGCAGCG
TGCAGCCTGAAGATGCAGCAACCTATTATTGTCAGCAGCGTAGCAGTTAT
CCGCTGACCTTTGGTGGTGGCACCAAACTGGAAATTAAAGGGGGTGGTGG
CTCAGGTGGCGGAGGTGCAGAAGCAGGTATTACCGGTACATGGTCAGATC
AGCTGGGTGATACCTTTATTGTTACCGCAGGCGCAGATGGTGCACTGACC
GGCACCTATGAAAATGCAGTTGGTGGTGCAGAAAGCCGTTATGTGCTGAC
CGGTCGTTATGATAGCGCACCGGCAACCGATGGTAGCGGCACCGCACTGG
GTTGGACCGTTGCATGGAAAAATAACAGCAAAAATGCACATAGCGCAACC
ACCTGGTCAGGTCAGTATGTGGGTGGTGCCGATGCCAAAATTAACACCCA
GTGGCTGCTGACCAGCGGTACAACCAATGCAAATGCCTGGAAAAGTACCC
TGGTTGGTCATGATACATTCACCAAAGTTAAACCGAGCGCAGCAAGCCAT
CATCATCACCATCATTAA
SEQ ID NO: 16
MKYLLPTAAAGLLLLAAQPAMAQVKLEQSGAEVVKPGASVKLSCKASGFN
IKDSYMHWLRQGPGQRLEWIGWIDPENGDTEYAPKFQGKATFTTDTSANT
AYLGLSSLRPEDTAVYYCNEGTPTGPYYFDWGQGTLVTVSSGGGGSGGGG
SGGGGSENVLTQSPSSMSVSVGDRVNIACSASSSVPYMHWLQQKPGKSPK
LLIYLTSNLASGVPSRFSGSGSGTDYSLTISSVQPEDAATYYCQQRSSYP
LTFGGGTKLEIKGGGGSGGGGAEAGITGTWSDQLGDTFIVTAGADGALTG
TYENAVGGAESRYVLTGRYDSAPATDGSGTALGWTVAWKNNSKNAHSATT
WSGQYVGGADAKINTQWLLTSGTTNANAWKSTLVGHDTFTKVKPSAASHH
HHHH*
SEQ ID NO: 17
ATGAAATATCTGCTGCCGACCGCAGCAGCGGGTCTGCTGCTGCTGGCAGC
ACAGCCTGCAATGGCACAGGTTAAACTGGAACAGAGCGGTGCCGAAGTTG
TTAAACCGGGTGCAAGCGTTAAACTGAGCTGTAAAGCAAGCGGCTTTAAC
ATCAAAGATAGCTATATGCATTGGCTGCGTCAGGGTCCGGGTCAGCGTCT
GGAATGGATTGGTTGGATTGATCCGGAAAATGGTGATACCGAATATGCAC
CGAAATTTVAGGGTAAAGCAACCTTTACCACCGATACCAGCGCAAATACC
GCATATCTGGGTCTGAGCAGCCTGCGTCCGGAAGATACCGCAGTGTATTA
TTGTAATGAAGGCACCCCGACCGGTCCGTATTATTTCGATTATTGGGGTC
AGGGCACCCTGGTTACCGTTAGCAGCGGTGGTGGTGGTAGTGGTGGCGGT
GGTTCAGGCGGTGGCGGTAGCGAAAATGTTCTGACCCAGAGCCCGAGCAG
CATGAGCGTTAGCGTTGGTGATCGTGTTAATATTGCATGTAGCGCAAGCA
GCAGCGTTCCGTACATGCACTGGCTGCAGCAGAAACCGGGTAAAAGCCCG
AAACTGCTGATTTATCTGACCAGCAATCTGGCAAGCGGTGTTCCGAGCCG
TTTTAGCGGTAGCGGTAGTGGCACCGATTATAGCCTGACCATTAGCAGCG
TGCAGCCTGAAGATGCAGCAACCTATTATTGTCAGCAGCGTAGCAGTTAT
CCGCTGACCTTTGGTGGTGGCACCAAACTGGAAATTAAAGGGGGTGGTGG
CTCAGGTGGCGGAGGTGCAGAAGCAGGTATTACCGGTACATGGTCAGATC
AGCTGGGTGATACCTTTATTGTTACCGCAGGCGCAGATGGTGCACTGACC
GGCACCTATGAAAATGCAGTTGGTGGTGCAGAAAGCCGTTATGTGCTGAC
CGGTCGTTATGATAGCGCACCGGCAACCGATGGTAGCGGCACCGCACTGG
GTTGGACCGTTGCATGGAAAAATAACAGCAAAAATGCACATAGCGCAACC
ACCTGGTCAGGTCAGTATGTGGGTGGTGCCGATGCCAAAATTAACACCCA
GTGGCTGCTGACCAGCGGTACAACCAATGCAAATGCCTGGAAAAGTACCC
TGGTTGGTCATGATACATTCACCAAAGTTAAATAA
SEQ ID NO: 18
MKYLLPTAAAGLLLLAAQPAMAQVKLEQSGAEVVKPGASVKLSCKASGFN
IKDSYMHWLRQGPGQRLEWIGWIDPENGDTEYAPKFQGKATFTTDTSANT
AYLGLSSLRPEDTAVYYCNEGTPTGPYYFDYWGQGTLVTVSSGGGGSGGG
GSGGGGSENVLTQSPSSMSVSVGDRVNIACSASSSVPYMHWLQQKPGKSP
KLLIYLTSNLASGVPSRFSGSGSGTDYSLTISSVQPEDAATYYCQQRSSY
PLTFGGGTKLEIKGGGGSGGGGAEAGITGTWSDQLGDTFIVTAGADGALT
GTYENAVGGAESRYVLTGRYDSAPATDGSGTALGWTVAWKNNSKNAHSAT
TWSGQYVGGADAKINTQWLLTSGTTNANAWKSTLVGHDTFTKVK*
SEQ ID NO: 19
AEAGITGTWSDQLGDTFIVTAGADGALTGTYENAVGGAESRYVLTGRYDS
APATDGSGTALGWTVAWKNNSKNAHSATTWSGQYVGGADAKINTQWLLTS
GTTNANAWKSTLVGHDTFTKVK*

| [SEQUENCE LISTING] | |
|---|---|
| SEQUENCE LISTING | |
| <110> Tokyo University | |
| <110> Savid Therapeutics Inc | |
| <120> Fused protein of an antibody which recognizes cancer- | |
| cells and a streptavidin mutant | |
| <130> 191762A | |
| <160> 19 | |
| <170> PatentIn version 3.5 | |
| <210> 1 | |
| <211> 133 | |
| <212> PRT | |
| <213> Artificial Sequence | |
| <220> | |
| <223> Description of Artificial Sequence: Recombinant protein | |
| <400> 1 | |
| Ala Glu Ala Gly Ile Thr Gly Thr Trp Ser Asp Gln Leu Gly Asp Thr | |
| 1 5 10 15 | |
| Phe Ile Val Thr Ala Gly Ala Asp Gly Ala Leu Thr Gly Thr Tyr Glu | |
| 20 25 30 | |
| Asn Ala Val Gly Gly Ala Glu Ser Arg Tyr Val Leu Thr Gly Arg Tyr | |
| 35 40 45 | |
| Asp Ser Ala Pro Ala Thr Asp Gly Ser Gly Thr Ala Leu Gly Trp Thr | |
| 50 55 60 | |
| Val Ala Trp Lys Asn Asn Ser Lys Asn Ala His Ser Ala Thr Thr Trp | |
| 65 70 75 80 | |
| Ser Gly Gln Tyr Val Gly Gly Ala Asp Ala Lys Ile Asn Thr Gln Trp | |
| 85 90 95 | |
| Leu Leu Thr Ser Gly Thr Thr Asn Ala Asn Ala Trp Lys Ser Thr Leu | |
| 100 105 110 | |
| Val Gly His Asp Thr Phe Thr Lys Val Lys Pro Ser Ala Ala Ser His | |
| 115 120 125 | |
| His His His His His | |
| 130 | |
| <210> 2 | |
| <211> 241 | |
| <212> PRT | |
| <213> Artificial Sequence | |
| <220> | |
| <223> Description of Artificial Sequence: Recombinant protein | |
| <400> 2 | |
| Gln Val Lys Leu Glu Gln Ser Gly Ala Glu Val Val Lys Pro Gly Ala | |
| 1 5 10 15 | |
| Ser Val Lys Leu Ser Cys Lys Ala Ser Gly Phe Asn Ile Lys Asp Ser | |
| 20 25 30 | |
| Tyr Met His Trp Leu Arg Gln Gly Pro Gly Gln Arg Leu Glu Trp Ile | |
| 35 40 45 | |
| Gly Trp Ile Asp Pro Glu Asn Gly Asp Thr Glu Tyr Ala Pro Lys Phe | |
| 50 55 60 | |
| Gln Gly Lys Ala Thr Phe Thr Thr Asp Thr Ser Ala Asn Thr Ala Tyr | |
| 65 70 75 80 | |
| Leu Gly Leu Ser Ser Leu Arg Pro Glu Asp Thr Ala Val Tyr Tyr Cys | |
| 85 90 95 | |
| Asn Glu Gly Thr Pro Thr Gly Pro Tyr Tyr Phe Asp Tyr Trp Gly Gln | |
| 100 105 110 | |
| Gly Thr Leu Val Thr Val Ser Ser Gly Gly Gly Gly Ser Gly Gly Gly | |
| 115 120 125 | |
| Gly Ser Gly Gly Gly Gly Ser Glu Asn Val Leu Thr Gln Ser Pro Ser | |
| 130 135 140 | |
| Ser Met Ser Val Ser Val Gly Asp Arg Val Asn Ile Ala Cys Ser Ala | |
| 145 150 155 160 | |
| Ser Ser Ser Val Pro Tyr Met His Trp Leu Gln Gln Lys Pro Gly Lys | |
| 165 170 175 | |
| Ser Pro Lys Leu Leu Ile Tyr Leu Thr Ser Asn Leu Ala Ser Gly Val | |
| 180 185 190 | |
| Pro Ser Arg Phe Ser Gly Ser Gly Ser Gly Thr Asp Tyr Ser Leu Thr | |
| 195 200 205 | |
| Ile Ser Ser Val Gln Pro Glu Asp Ala Ala Thr Tyr Tyr Cys Gln Gln | |
| 210 215 220 | |
| Arg Ser Ser Tyr Pro Leu Thr Phe Gly Gly Gly Thr Lys Leu Glu Ile | |
| 225 230 235 240 | |
| Lys | |
| <210> 3 | |
| <211> 383 | |
| <212> PRT | |
| <213> Artificial Sequence | |
| <220> | |
| <223> Description of Artificial Sequence: Recombinant protein | |
| <400> 3 | |
| Gln Val Lys Leu Glu Gln Ser Gly Ala Glu Val Val Lys Pro Gly Ala | |
| 1 5 10 15 | |
| Ser Val Lys Leu Ser Cys Lys Ala Ser Gly Phe Asn Ile Lys Asp Ser | |
| 20 25 30 | |
| Tyr Met His Trp Leu Arg Gln Gly Pro Gly Gln Arg Leu Glu Trp Ile | |
| 35 40 45 | |
| Gly Trp Ile Asp Pro Glu Asn Gly Asp Thr Glu Tyr Ala Pro Lys Phe | |
| 50 55 60 | |
| Gln Gly Lys Ala Thr Phe Thr Thr Asp Thr Ser Ala Asn Thr Ala Tyr | |
| 65 70 75 80 | |
| Leu Gly Leu Ser Ser Leu Arg Pro Glu Asp Thr Ala Val Tyr Tyr Cys | |
| 85 90 95 | |
| Asn Glu Gly Thr Pro Thr Gly Pro Tyr Tyr Phe Asp Tyr Trp Gly Gln | |
| 100 105 110 | |
| Gly Thr Leu Val Thr Val Ser Ser Gly Gly Gly Gly Ser Gly Gly Gly | |
| 115 120 125 | |
| Gly Ser Gly Gly Gly Gly Ser Glu Asn Val Leu Thr Gln Ser Pro Ser | |
| 130 135 140 | |
| Ser Met Ser Val Ser Val Gly Asp Arg Val Asn Ile Ala Cys Ser Ala | |
| 145 150 155 160 | |
| Ser Ser Ser Val Pro Tyr Met His Trp Leu Gln Gln Lys Pro Gly Lys | |
| 165 170 175 | |
| Ser Pro Lys Leu Leu Ile Tyr Leu Thr Ser Asn Leu Ala Ser Gly Val | |
| 180 185 190 | |
| Pro Ser Arg Phe Ser Gly Ser Gly Ser Gly Thr Asp Tyr Ser Leu Thr | |
| 195 200 205 | |
| Ile Ser Ser Val Gln Pro Glu Asp Ala Ala Thr Tyr Tyr Cys Gln Gln | |
| 210 215 220 | |
| Arg Ser Ser Tyr Pro Leu Thr Phe Gly Gly Gly Thr Lys Leu Glu Ile | |
| 225 230 235 240 | |
| Lys Gly Gly Gly Gly Ser Gly Gly Gly Gly Ala Glu Ala Gly Ile Thr | |
| 245 250 255 | |
| Gly Thr Trp Ser Asp Gln Leu Gly Asp Thr Phe Ile Val Thr Ala Gly | |
| 260 265 270 | |
| Ala Asp Gly Ala Leu Thr Gly Thr Tyr Glu Asn Ala Val Gly Gly Ala | |
| 275 280 285 | |
| Glu Ser Arg Tyr Val Leu Thr Gly Arg Tyr Asp Ser Ala Pro Ala Thr | |
| 290 295 300 | |
| Asp Gly Ser Gly Thr Ala Leu Gly Trp Thr Val Ala Trp Lys Asn Asn | |
| 305 310 315 320 | |
| Ser Lys Asn Ala His Ser Ala Thr Thr Trp Ser Gly Gln Tyr Val Gly | |
| 325 330 335 | |
| Gly Ala Asp Ala Lys Ile Asn Thr Gln Trp Leu Leu Thr Ser Gly Thr | |
| 340 345 350 | |
| Thr Asn Ala Asn Ala Trp Lys Ser Thr Leu Val Gly His Asp Thr Phe | |
| 355 360 365 | |
| Thr Lys Val Lys Pro Ser Ala Ala Ser His His His His His His | |
| 370 375 380 | |
| <210> 4 | |
| <211> 405 | |
| <212> PRT | |
| <213> Artificial Sequence | |
| <220> | |
| <223> Description of Artificial Sequence: Recombinant protein | |
| <400> 4 | |
| Met Lys Tyr Leu Leu Pro Thr Ala Ala Ala Gly Leu Leu Leu Leu Ala | |
| 1 5 10 15 | |
| Ala Gln Pro Ala Met Ala Gln Val Lys Leu Glu Gln Ser Gly Ala Glu | |
| 20 25 30 | |
| Val Val Lys Pro Gly Ala Ser Val Lys Leu Ser Cys Lys Ala Ser Gly | |
| 35 40 45 | |
| Phe Asn Ile Lys Asp Ser Tyr Met His Trp Leu Arg Gln Gly Pro Gly | |
| 50 55 60 | |
| Gln Arg Leu Glu Trp Ile Gly Trp Ile Asp Pro Glu Asn Gly Asp Thr | |
| 65 70 75 80 | |
| Glu Tyr Ala Pro Lys Phe Gln Gly Lys Ala Thr Phe Thr Thr Asp Thr | |
| 85 90 95 | |
| Ser Ala Asn Thr Ala Tyr Leu Gly Leu Ser Ser Leu Arg Pro Glu Asp | |
| 100 105 110 | |
| Thr Ala Val Tyr Tyr Cys Asn Glu Gly Thr Pro Thr Gly Pro Tyr Tyr | |
| 115 120 125 | |
| Phe Asp Tyr Trp Gly Gln Gly Thr Leu Val Thr Val Ser Ser Gly Gly | |
| 130 135 140 | |
| Gly Gly Ser Gly Gly Gly Gly Ser Gly Gly Gly Gly Ser Glu Asn Val | |
| 145 150 155 160 | |
| Leu Thr Gln Ser Pro Ser Ser Met Ser Val Ser Val Gly Asp Arg Val | |
| 165 170 175 | |
| Asn Ile Ala Cys Ser Ala Ser Ser Ser Val Pro Tyr Met His Trp Leu | |
| 180 185 190 | |
| Gln Gln Lys Pro Gly Lys Ser Pro Lys Leu Leu Ile Tyr Leu Thr Ser | |
| 195 200 205 | |
| Asn Leu Ala Ser Gly Val Pro Ser Arg Phe Ser Gly Ser Gly Ser Gly | |
| 210 215 220 | |
| Thr Asp Tyr Ser Leu Thr Ile Ser Ser Val Gln Pro Glu Asp Ala Ala | |
| 225 230 235 240 | |
| Thr Tyr Tyr Cys Gln Gln Arg Ser Ser Tyr Pro Leu Thr Phe Gly Gly | |
| 245 250 255 | |
| Gly Thr Lys Leu Glu Ile Lys Gly Gly Gly Gly Ser Gly Gly Gly Gly | |
| 260 265 270 | |
| Ala Glu Ala Gly Ile Thr Gly Thr Trp Ser Asp Gln Leu Gly Asp Thr | |
| 275 280 285 | |
| Phe Ile Val Thr Ala Gly Ala Asp Gly Ala Leu Thr Gly Thr Tyr Glu | |
| 290 295 300 | |
| Asn Ala Val Gly Gly Ala Glu Ser Arg Tyr Val Leu Thr Gly Arg Tyr | |
| 305 310 315 320 | |
| Asp Ser Ala Pro Ala Thr Asp Gly Ser Gly Thr Ala Leu Gly Trp Thr | |
| 325 330 335 | |
| Val Ala Trp Lys Asn Asn Ser Lys Asn Ala His Ser Ala Thr Thr Trp | |
| 340 345 350 | |
| Ser Gly Gln Tyr Val Gly Gly Ala Asp Ala Lys Ile Asn Thr Gln Trp | |
| 355 360 365 | |
| Leu Leu Thr Ser Gly Thr Thr Asn Ala Asn Ala Trp Lys Ser Thr Leu | |
| 370 375 380 | |
| Val Gly His Asp Thr Phe Thr Lys Val Lys Pro Ser Ala Ala Ser His | |
| 385 390 395 400 | |
| His His His His His | |
| <210> 5 | |
| <211> 1218 | |
| <212> DNA | |
| <213> Artificial Sequence | |
| <220> | |
| <223> Description of Artificial Sequence: Recombinant DNA | |
| <400> 5 | |
| atgaaatatc tgctgccgac cgcagcagcg ggtctgctgc tgctggcagc acagcctgca | 60 |
| atggcacagg ttaaactgga acagagcggt gccgaagttg ttaaaccggg tgcaagcgtt | 120 |
| aaactgagct gtaaagcaag cggctttaac atcaaagata gctatatgca ttggctgcgt | 180 |
| cagggtccgg gtcagcgtct ggaatggatt ggttggattg atccggaaaa tggtgatacc | 240 |
| gaatatgcac cgaaatttca gggtaaagca acctttacca ccgataccag cgcaaatacc | 300 |
| gcatatctgg gtctgagcag cctgcgtccg gaagataccg cagtgtatta ttgtaatgaa | 360 |
| ggcaccccga ccggtccgta ttatttcgat tattggggtc agggcaccct ggttaccgtt | 420 |
| agcagcggtg gtggtggtag tggtggcggt ggttcaggcg gtggcggtag cgaaaatgtt | 480 |
| ctgacccaga gcccgagcag catgagcgtt agcgttggtg atcgtgttaa tattgcatgt | 540 |
| agcgcaagca gcagcgttcc gtacatgcac tggctgcagc agaaaccggg taaaagcccg | 600 |
| aaactgctga tttatctgac cagcaatctg gcaagcggtg ttccgagccg ttttagcggt | 660 |
| agcggtagtg gcaccgatta tagcctgacc attagcagcg tgcagcctga agatgcagca | 720 |
| acctattatt gtcagcagcg tagcagttat ccgctgacct ttggtggtgg caccaaactg | 780 |
| gaaattaaag ggggtggtgg ctcaggtggc ggaggtgcag aagcaggtat taccggtaca | 840 |
| tggtcagatc agctgggtga tacctttatt gttaccgcag gcgcagatgg tgcactgacc | 900 |
| ggcacctatg aaaatgcagt tggtggtgca gaaagccgtt atgtgctgac cggtcgttat | 960 |
| gatagcgcac cggcaaccga tggtagcggc accgcactgg gttggaccgt tgcatggaaa | 1020 |
| aataacagca aaaatgcaca tagcgcaacc acctggtcag gtcagtatgt gggtggtgcc | 1080 |
| gatgccaaaa ttaacaccca gtggctgctg accagcggta caaccaatgc aaatgcctgg | 1140 |
| aaaagtaccc tggttggtca tgatacattc accaaagtta aaccgagcgc agcaagccat | 1200 |
| catcatcacc atcattaa | 1218 |
| <210> 6 | |
| <211> 9 | |
| <212> PRT | |
| <213> Artificial Sequence | |
| <220> | |
| <223> Description of Artificial Sequence: Recombinant linker | |
| <400> 6 | |
| Gly Gly Gly Gly Ser Gly Gly Gly Gly | |
| 1 5 | |
| <210> 7 | |
| <211> 40 | |
| <212> DNA | |
| <213> Artificial Sequence | |
| <220> | |
| <223> Description of Artificial Sequence: Synthesized DNA | |
| <400> 7 | |
| aaggagatat acatatggat aaaattgcca ttgttaatat | 40 |
| <210> 8 | |
| <211> 39 | |
| <212> DNA | |
| <213> Artificial Sequence | |
| <220> | |
| <223> Description of Artificial Sequence: Synthesized DNA | |
| <400> 8 | |
| ttgagatctg ccatatgtta tttcacttgt ttcagaacg | 39 |
| <210> 9 | |
| <211> 35 | |
| <212> DNA | |
| <213> Artificial Sequence | |
| <220> | |
| <223> Description of Artificial Sequence: Synthesized DNA | |
| <400> 9 | |
| agaaggagat ataccatgaa atatctgctg ccgac | 35 |
| <210> 10 | |
| <211> 36 | |
| <212> DNA | |
| <213> Artificial Sequence | |
| <220> | |
| <223> Description of Artificial Sequence: Synthesized DNA | |
| <400> 10 | |
| cgccgagctc gaattttaat gatggtgatg atgatg | 36 |
| <210> 11 | |
| <211> 26 | |
| <212> DNA | |
| <213> Artificial Sequence | |
| <220> | |
| <223> Description of Artificial Sequence: Synthesized DNA | |
| <400> 11 | |
| ggtatatctc cttcttaaag ttaaac | 26 |
| <210> 12 | |
| <211> 24 | |
| <212> DNA | |
| <213> Artificial Sequence | |
| <220> | |
| <223> Description of Artificial Sequence: Synthesized DNA | |
| <400> 12 | |
| aattcgagct cggcgcgcct gcag | 24 |
| <210> 13 | |
| <211> 27 | |
| <212> DNA | |
| <213> Artificial Sequence | |
| <220> | |
| <223> Description of Artificial Sequence: Synthesized DNA | |
| <400> 13 | |
| agttaaataa cgagctcggc gcgcctg | 27 |
| <210> 14 | |
| <211> 29 | |
| <212> DNA | |
| <213> Artificial Sequence | |
| <220> | |
| <223> Description of Artificial Sequence: Synthesized DNA | |
| <400> 14 | |
| gctcgttatt taactttggt gaatgtatc | 29 |
| <210> 15 | |
| <211> 1218 | |
| <212> DNA | |
| <213> Artificial Sequence | |
| <220> | |
| <223> Description of Artificial Sequence: Recombinant DNA | |
| <400> 15 | |
| atgaaatatc tgctgccgac cgcagcagcg ggtctgctgc tgctggcagc acagcctgca | 60 |
| atggcacagg ttaaactgga acagagcggt gccgaagttg ttaaaccggg tgcaagcgtt | 120 |
| aaactgagct gtaaagcaag cggctttaac atcaaagata gctatatgca ttggctgcgt | 180 |
| cagggtccgg gtcagcgtct ggaatggatt ggttggattg atccggaaaa tggtgatacc | 240 |
| gaatatgcac cgaaatttca gggtaaagca acctttacca ccgataccag cgcaaatacc | 300 |
| gcatatctgg gtctgagcag cctgcgtccg gaagataccg cagtgtatta ttgtaatgaa | 360 |
| ggcaccccga ccggtccgta ttatttcgat tattggggtc agggcaccct ggttaccgtt | 420 |
| agcagcggtg gtggtggtag tggtggcggt ggttcaggcg gtggcggtag cgaaaatgtt | 480 |
| ctgacccaga gcccgagcag catgagcgtt agcgttggtg atcgtgttaa tattgcatgt | 540 |
| agcgcaagca gcagcgttcc gtacatgcac tggctgcagc agaaaccggg taaaagcccg | 600 |
| aaactgctga tttatctgac cagcaatctg gcaagcggtg ttccgagccg ttttagcggt | 660 |
| agcggtagtg gcaccgatta tagcctgacc attagcagcg tgcagcctga agatgcagca | 720 |
| acctattatt gtcagcagcg tagcagttat ccgctgacct ttggtggtgg caccaaactg | 780 |
| gaaattaaag ggggtggtgg ctcaggtggc ggaggtgcag aagcaggtat taccggtaca | 840 |
| tggtcagatc agctgggtga tacctttatt gttaccgcag gcgcagatgg tgcactgacc | 900 |
| ggcacctatg aaaatgcagt tggtggtgca gaaagccgtt atgtgctgac cggtcgttat | 960 |
| gatagcgcac cggcaaccga tggtagcggc accgcactgg gttggaccgt tgcatggaaa | 1020 |
| aataacagca aaaatgcaca tagcgcaacc acctggtcag gtcagtatgt gggtggtgcc | 1080 |
| gatgccaaaa ttaacaccca gtggctgctg accagcggta caaccaatgc aaatgcctgg | 1140 |
| aaaagtaccc tggttggtca tgatacattc accaaagtta aaccgagcgc agcaagccat | 1200 |
| catcatcacc atcattaa | 1218 |
| <210> 16 | |
| <211> 405 | |
| <212> PRT | |
| <213> Artificial Sequence | |
| <220> | |
| <223> Description of Artificial Sequence: Recombinant protein | |
| <400> 16 | |
| Met Lys Tyr Leu Leu Pro Thr Ala Ala Ala Gly Leu Leu Leu Leu Ala | |
| 1 5 10 15 | |
| Ala Gln Pro Ala Met Ala Gln Val Lys Leu Glu Gln Ser Gly Ala Glu | |
| 20 25 30 | |
| Val Val Lys Pro Gly Ala Ser Val Lys Leu Ser Cys Lys Ala Ser Gly | |
| 35 40 45 | |
| Phe Asn Ile Lys Asp Ser Tyr Met His Trp Leu Arg Gln Gly Pro Gly | |
| 50 55 60 | |
| Gln Arg Leu Glu Trp Ile Gly Trp Ile Asp Pro Glu Asn Gly Asp Thr | |
| 65 70 75 80 | |
| Glu Tyr Ala Pro Lys Phe Gln Gly Lys Ala Thr Phe Thr Thr Asp Thr | |
| 85 90 95 | |
| Ser Ala Asn Thr Ala Tyr Leu Gly Leu Ser Ser Leu Arg Pro Glu Asp | |
| 100 105 110 | |
| Thr Ala Val Tyr Tyr Cys Asn Glu Gly Thr Pro Thr Gly Pro Tyr Tyr | |
| 115 120 125 | |
| Phe Asp Tyr Trp Gly Gln Gly Thr Leu Val Thr Val Ser Ser Gly Gly | |
| 130 135 140 | |
| Gly Gly Ser Gly Gly Gly Gly Ser Gly Gly Gly Gly Ser Glu Asn Val | |
| 145 150 155 160 | |
| Leu Thr Gln Ser Pro Ser Ser Met Ser Val Ser Val Gly Asp Arg Val | |
| 165 170 175 | |
| Asn Ile Ala Cys Ser Ala Ser Ser Ser Val Pro Tyr Met His Trp Leu | |
| 180 185 190 | |
| Gln Gln Lys Pro Gly Lys Ser Pro Lys Leu Leu Ile Tyr Leu Thr Ser | |
| 195 200 205 | |
| Asn Leu Ala Ser Gly Val Pro Ser Arg Phe Ser Gly Ser Gly Ser Gly | |
| 210 215 220 | |
| Thr Asp Tyr Ser Leu Thr Ile Ser Ser Val Gln Pro Glu Asp Ala Ala | |
| 225 230 235 240 | |
| Thr Tyr Tyr Cys Gln Gln Arg Ser Ser Tyr Pro Leu Thr Phe Gly Gly | |
| 245 250 255 | |
| Gly Thr Lys Leu Glu Ile Lys Gly Gly Gly Gly Ser Gly Gly Gly Gly | |
| 260 265 270 | |
| Ala Glu Ala Gly Ile Thr Gly Thr Trp Ser Asp Gln Leu Gly Asp Thr | |
| 275 280 285 | |
| Phe Ile Val Thr Ala Gly Ala Asp Gly Ala Leu Thr Gly Thr Tyr Glu | |
| 290 295 300 | |
| Asn Ala Val Gly Gly Ala Glu Ser Arg Tyr Val Leu Thr Gly Arg Tyr | |
| 305 310 315 320 | |
| Asp Ser Ala Pro Ala Thr Asp Gly Ser Gly Thr Ala Leu Gly Trp Thr | |
| 325 330 335 | |
| Val Ala Trp Lys Asn Asn Ser Lys Asn Ala His Ser Ala Thr Thr Trp | |
| 340 345 350 | |
| Ser Gly Gln Tyr Val Gly Gly Ala Asp Ala Lys Ile Asn Thr Gln Trp | |
| 355 360 365 | |
| Leu Leu Thr Ser Gly Thr Thr Asn Ala Asn Ala Trp Lys Ser Thr Leu | |
| 370 375 380 | |
| Val Gly His Asp Thr Phe Thr Lys Val Lys Pro Ser Ala Ala Ser His | |
| 385 390 395 400 | |
| His His His His His | |
| 405 | |
| <210> 17 | |
| <211> 1185 | |
| <212> DNA | |
| <213> Artificial Sequence | |
| <220> | |
| <223> Description of Artificial Sequence: Recombinant DNA | |
| <400> 17 | |
| atgaaatatc tgctgccgac cgcagcagcg ggtctgctgc tgctggcagc acagcctgca | 60 |
| atggcacagg ttaaactgga acagagcggt gccgaagttg ttaaaccggg tgcaagcgtt | 120 |
| aaactgagct gtaaagcaag cggctttaac atcaaagata gctatatgca ttggctgcgt | 180 |
| cagggtccgg gtcagcgtct ggaatggatt ggttggattg atccggaaaa tggtgatacc | 240 |
| gaatatgcac cgaaatttca gggtaaagca acctttacca ccgataccag cgcaaatacc | 300 |
| gcatatctgg gtctgagcag cctgcgtccg gaagataccg cagtgtatta ttgtaatgaa | 360 |
| ggcaccccga ccggtccgta ttatttcgat tattggggtc agggcaccct ggttaccgtt | 420 |
| agcagcggtg gtggtggtag tggtggcggt ggttcaggcg gtggcggtag cgaaaatgtt | 480 |
| ctgacccaga gcccgagcag catgagcgtt agcgttggtg atcgtgttaa tattgcatgt | 540 |
| agcgcaagca gcagcgttcc gtacatgcac tggctgcagc agaaaccggg taaaagcccg | 600 |
| aaactgctga tttatctgac cagcaatctg gcaagcggtg ttccgagccg ttttagcggt | 660 |
| agcggtagtg gcaccgatta tagcctgacc attagcagcg tgcagcctga agatgcagca | 720 |
| acctattatt gtcagcagcg tagcagttat ccgctgacct ttggtggtgg caccaaactg | 780 |
| gaaattaaag ggggtggtgg ctcaggtggc ggaggtgcag aagcaggtat taccggtaca | 840 |
| tggtcagatc agctgggtga tacctttatt gttaccgcag gcgcagatgg tgcactgacc | 900 |
| ggcacctatg aaaatgcagt tggtggtgca gaaagccgtt atgtgctgac cggtcgttat | 960 |
| gatagcgcac cggcaaccga tggtagcggc accgcactgg gttggaccgt tgcatggaaa | 1020 |
| aataacagca aaaatgcaca tagcgcaacc acctggtcag gtcagtatgt gggtggtgcc | 1080 |
| gatgccaaaa ttaacaccca gtggctgctg accagcggta caaccaatgc aaatgcctgg | 1140 |
| aaaagtaccc tggttggtca tgatacattc accaaagtta aataa | 1185 |

```
<210> 18
<211> 394
<212> PRT
<213> Artificial Sequence
<220>
<223> Description of Artificial Sequence: Recombinant protein
<400> 18
Met Lys Tyr Leu Leu Pro Thr Ala Ala Ala Gly Leu Leu Leu Leu Ala
1 5 10 15
Ala Gln Pro Ala Met Ala Gln Val Lys Leu Glu Gln Ser Gly Ala Glu
                        20 25 30
Val Val Lys Pro Gly Ala Ser Val Lys Leu Ser Cys Lys Ala Ser Gly
                35 40 45
Phe Asn Ile Lys Asp Ser Tyr Met His Trp Leu Arg Gln Gly Pro Gly
        50 55 60
Gln Arg Leu Glu Trp Ile Gly Trp Ile Asp Pro Glu Asn Gly Asp Thr
65 70 75 80
Glu Tyr Ala Pro Lys Phe Gln Gly Lys Ala Thr Phe Thr Thr Asp Thr
                                85 90 95
Ser Ala Asn Thr Ala Tyr Leu Gly Leu Ser Ser Leu Arg Pro Glu Asp
                        100 105 110
Thr Ala Val Tyr Tyr Cys Asn Glu Gly Thr Pro Thr Gly Pro Tyr Tyr
                115 120 125
Phe Asp Tyr Trp Gly Gln Gly Thr Leu Val Thr Val Ser Ser Gly Gly
        130 135 140
Gly Gly Ser Gly Gly Gly Gly Ser Gly Gly Gly Gly Ser Glu Asn Val
145 150 155 160
Leu Thr Gln Ser Pro Ser Ser Met Ser Val Ser Val Gly Asp Arg Val
                                165 170 175
Asn Ile Ala Cys Ser Ala Ser Ser Ser Val Pro Tyr Met His Trp Leu
                        180 185 190
Gln Gln Lys Pro Gly Lys Ser Pro Lys Leu Leu Ile Tyr Leu Thr Ser
                195 200 205
Asn Leu Ala Ser Gly Val Pro Ser Arg Phe Ser Gly Ser Gly Ser Gly
        210 215 220
Thr Asp Tyr Ser Leu Thr Ile Ser Ser Val Gln Pro Glu Asp Ala Ala
225 230 235 240
Thr Tyr Tyr Cys Gln Gln Arg Ser Ser Tyr Pro Leu Thr Phe Gly Gly
                                245 250 255
Gly Thr Lys Leu Glu Ile Lys Gly Gly Gly Gly Ser Gly Gly Gly Gly
                        260 265 270
Ala Glu Ala Gly Ile Thr Gly Thr Trp Ser Asp Gln Leu Gly Asp Thr
                275 280 285
Phe Ile Val Thr Ala Gly Ala Asp Gly Ala Leu Thr Gly Thr Tyr Glu
        290 295 300
Asn Ala Val Gly Gly Ala Glu Ser Arg Tyr Val Leu Thr Gly Arg Tyr
305 310 315 320
Asp Ser Ala Pro Ala Thr Asp Gly Ser Gly Thr Ala Leu Gly Trp Thr
                                325 330 335
Val Ala Trp Lys Asn Asn Ser Lys Asn Ala His Ser Ala Thr Thr Trp
                        340 345 350
Ser Gly Gln Tyr Val Gly Gly Ala Asp Ala Lys Ile Asn Thr Gln Trp
                355 360 365
Leu Leu Thr Ser Gly Thr Thr Asn Ala Asn Ala Trp Lys Ser Thr Leu
        370 375 380
Val Gly His Asp Thr Phe Thr Lys Val Lys
385 390
<210> 19
<211> 122
<212> PRT
<213> Artificial Sequence
<220>
<223> Description of Artificial Sequence: Recombinant protein
<400> 19
Ala Glu Ala Gly Ile Thr Gly Thr Trp Ser Asp Gln Leu Gly Asp Thr
1 5 10 15
Phe Ile Val Thr Ala Gly Ala Asp Gly Ala Leu Thr Gly Thr Tyr Glu
                        20 25 30
Asn Ala Val Gly Gly Ala Glu Ser Arg Tyr Val Leu Thr Gly Arg Tyr
                35 40 45
Asp Ser Ala Pro Ala Thr Asp Gly Ser Gly Thr Ala Leu Gly Trp Thr
        50 55 60
Val Ala Trp Lys Asn Asn Ser Lys Asn Ala His Ser Ala Thr Thr Trp
65 70 75 80
Ser Gly Gln Tyr Val Gly Gly Ala Asp Ala Lys Ile Asn Thr Gln Trp
                                85 90 95
Leu Leu Thr Ser Gly Thr Thr Asn Ala Asn Ala Trp Lys Ser Thr Leu
                        100 105 110
Val Gly His Asp Thr Phe Thr Lys Val Lys
                115 120
```

1. A fusion protein comprising a protein-recognizing substance and the amino acid sequence as set forth in SEQ ID NO: 19 that is fused with the protein-recognizing substance.

2. The fusion protein according to claim 1, wherein the protein-recognizing substance is a substance that recognizes cancer cells.

3. The fusion protein according to claim 2, wherein the antibody that recognizes cancer cells is an anti-CEA antibody or an anti-HER2 antibody.

4. The fusion protein according to claim 1, which has the amino acid sequence as set forth in SEQ ID NO: 18.

5. A nucleic acid encoding the amino acid sequence as set forth in SEQ ID NO: 18.

6. A cancer therapeutic agent or a cancer diagnostic agent, comprising the fusion protein according to claim 1.

7. A kit for treating or diagnosing cancer, comprising (1) the fusion protein according to claim 1, and (2) a conjugate of a compound represented by the following formula (1) or a salt thereof and a diagnostic substance or a therapeutic substance: 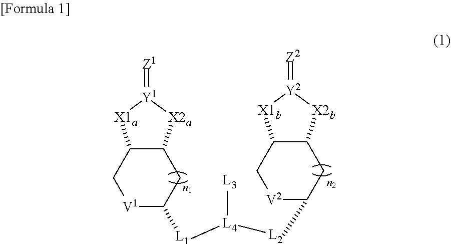
wherein
X1a, X1b, X2a and X2b each independently represent O or NH,
Y¹ and Y² each independently represent C or S,
Z¹ and Z² each independently represent O, S or NH,
V¹ and V² each independently represent S or S⁺—O⁻,
n1 and n2 each independently represent an integer of O or 1,
L₁ and L₂ each independently represent a divalent linking group,
L₃ represents a group comprising a functional group capable of binding to the diagnostic substance or the therapeutic substance at the terminus, and
L₄ represents a trivalent linking group.

8. The kit according to claim 7, wherein the diagnostic substance or the therapeutic substance is a phthalocyanine dye.
